(19) United States
(12) Patent Application Publication
(10) Pub. No.: US 2017/0191973 A1
(43) Pub. Date: Jul. 6, 2017

(54) DISTRIBUTED BIOLOGICAL MONITORING SYSTEM

(71) Applicants: Christopher A. Eusebi, Bloomfield Twp., MI (US); Brent M. Nowak, Ada, MI (US); Barry D. Nowak, Grand Rapids, MI (US)

(72) Inventors: Christopher A. Eusebi, Bloomfield Twp., MI (US); Brent M. Nowak, Ada, MI (US); Barry D. Nowak, Grand Rapids, MI (US)

(21) Appl. No.: 15/291,413

(22) Filed: Oct. 12, 2016

Related U.S. Application Data
(60) Provisional application No. 62/240,798, filed on Oct. 13, 2015.

(51) Int. Cl. G01N 33/00; G06F 19/00; G01N 33/50

(52) U.S. Cl. CPC G01N 33/0075; G01N 33/0031; G01N 33/0047; G01N 33/0062; G01N 33/5097; G06F 19/3493

(57) ABSTRACT

A system for the detection of biological markers over a distributed area is disclosed. The system utilizes a fleet of mobile platforms, which are pseudo randomly distributed over a geographic region. The mobile platforms are equipped with an indexable air filter system, which captures samples of spores, pollen, or dust at discrete geographic or temporal nodes. The nodes are programmable, which allows both temporal and geographic data points to be linear, geometric, exponential or other non-linear modes that allows for unique analysis and data compression algorithms.

This application claims the benefit of U.S. Provisional Application No. 62/240,798, filed on Oct. 13, 2015. The entire disclosure of the above application is incorporated herein by reference.

### FIELD

The present disclosure relates generally to a method and system for detecting chemical, radiological and/or biological agents and, more particularly, to a method and system for detecting air or fluid borne chemical, radiological and/or biological agents using a filter system on a plurality of mobile platforms. The system uniquely characterizes the specimens simultaneously in geographic, environment and temporal modes.

### BACKGROUND

This section provides background information related to the present disclosure, which is not necessarily prior art. The modeling of agricultural natural biological systems is difficult due to the lack of reliable systems to monitor and validate data. Whether it is the spread of genetically modified organisms from natural pollen transmission, or the natural ebbs and flows of naturally occurring fungal parasites, the monitoring of such systems is difficult due to the large land areas and associated weather patterns. The accuracy of these models is important to determine the predictability of crop yields and to assist in the interception of pests or biological threats.

### SUMMARY

This section provides a general summary of the disclosure, and is not a comprehensive disclosure of its full scope or all of its features. Early warnings and ongoing characterization and modeling of agricultural disease outbreaks require effective systems to monitor the spread of biological markers in real-time. The central element of bio-surveillance or a nation's food supply is the detection and monitoring of herb-logical and mycological biological markers within a distributed environment.

According to the teachings of one embodiment, a system is provided to evaluate the distributed monitoring of biological markers within a geographic region. The system is capable of near real-time monitoring of biological markers and the food supply. Generally, the system utilizes a fleet of mobile platforms, which are pseudo randomly distributed over a geographic region. The mobile platforms are equipped with an indexable air filter system, which captures samples of spores, pollen, or dust at discrete geographic or temporal nodes. The nodes are programmable, which allows both temporal and geographic data points to be linear, geometric, exponential or other non-linear modes that allows for unique analysis and data compression algorithms. Geographic location (e.g., latitude, longitude, altitude), time and atmospherics (e.g., wind, humidity, ambient light) of each sample are also collected. Uniquely, this embodiment allows for the system to respond to environmental conditions (weather, altitude, geography, etc). That is, the system senses whether the conditions are appropriate for sampling, and may be triggered by spores, pollen, dust, radiological, or biologics to increase or decrease the sampling rate.

General amplifications such as real-time PCR and PCR techniques combined with gel electrophoresis can then be used to analyze each sample to determine the existence or quantity of biological markers at a specific location or locations. Data from these systems can then be used to validate and improve computer modeling describing the spread of biological markers.

According to the present teachings, a system for detecting the spread of biological agents is disclosed. This system utilizes an array of filter modules, each placed on a mobile platform. The filter modules have an associated electronic tracking module configured to monitor the movement of the filter module within a predetermined space. The filter modules have a filter media configured to take several discrete samples at varying locations within the geographic region.

According to another aspect of the teachings, a filter system for detecting biological agents is described. The filter system has a vacuum source coupled to the filter media. An indexable aperture is provided which is moved in relation to the filter media to allow discrete portions of the filter to be exposed to a stream of fluid or air being monitored. A tracking device is provided which monitors the location of the filter in space and time. Locational information is then correlated to discrete samples, which are taken on the filter media. Uniquely this system accounts for and adapts to atmospheric and environmental conditions that play critical roles in the distribution, lifting, and deposition of spores, seeds, biologics, radioisotopes, and other.

According to another aspect of the teachings, the filter system described above has a tracking device, which provides at least one of time or location correlations to the discrete positions of the filter media.

According to another aspect of the teachings, a plurality of mobile platforms travelling within a predetermined geographic region are provided. Each mobile platform has a filter system as described above. The filter system has an indexable filter media. Air being sampled is pulled through at least one discrete location on the filter media. A geographic location or locations and optionally with time and environmental conditions associated with individual samples is then associated within a memory storage device. Collectively, in this embodiment, the fleet of vehicles may be provided with wireless, GPS, or other communication modes, which allow the collection and correlation to be adapted in real-time across broad geographic areas, or temporal modes. This fleet of filters are subsumed into a larger system-of-systems that provide another novel and improved sensing, measurement, and analysis.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 represents a system of distributed mobile platforms according to the present teachings;

FIG. 2 represents a vehicle equipped with an indexable filter media according to the present teachings;

FIGS. 3a and 3b represent indexable filter systems according to the present teachings;

FIGS. 4a and 4b represent exploded views of the indexable media shown in FIGS. 3a and 3b;

FIG. 5 represents a perspective view alternate filter system according to the present teachings;

FIG. 6 represents a top view of indexable filter media shown in FIG. 5; and

FIG. 7 represents a map showing the acquisition and detection of biological markers using the system described above.

Corresponding reference numerals indicate corresponding parts throughout the several views of the drawings.

### DETAILED DESCRIPTION

Example embodiments will now be described more fully with reference to the accompanying drawings.

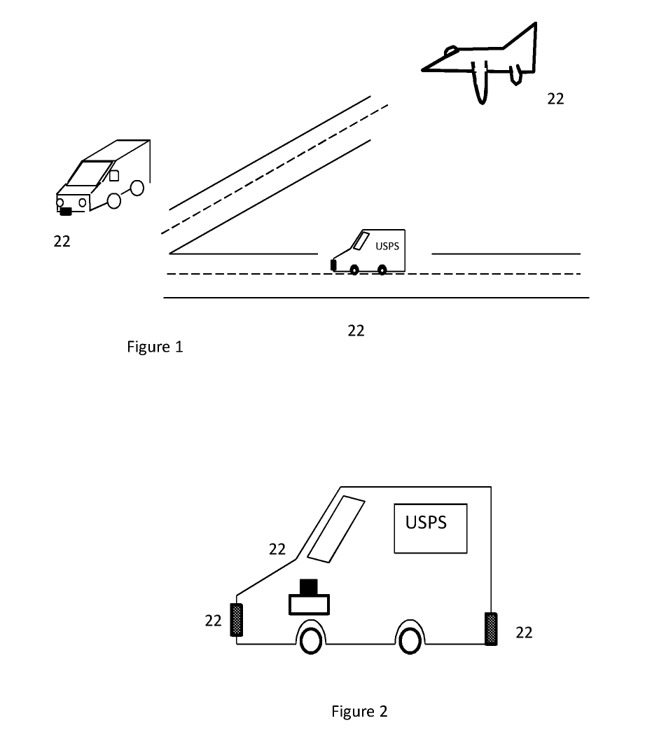

FIG. 1 represents a system for the detection of biological, chemical and/or radiological markers within a geographic region. The system utilizes a fleet of mobile platforms 20 having a detection module 22. The vehicles within the fleet may communicate local conditions and sampling history; may reference onboard or remote databases to adapt to changing conditions; or may receive predictive atmospheric conditions to appropriately sample prior to wind changes, rain, or pressure drops, as well as during and after meteorological events. The detection module 22 is configured to take samples of biological, chemical, and/or radiological materials at locations within a geographic region. The module 22 is configured to additionally collect temporal data related to the location, and correlate the locations of each sampling location within the geographic location. Additionally, the module correlates the location of the sample on the filter medium to the sampling location as expressed as the vehicle location.

The fleet of mobile platforms 20 can be a fleet of water, airborne, or land vehicles such as automobiles or trucks. The vehicles can take the form of a governmental or non-governmental delivery vehicle such as U.S. Postal Service or any number of commercial delivery vehicle organizations. As described in more detail below, the nature of the commercial or governmental delivery system allows for a pseudo-random sampling or sampling at predetermined locations along a road system within a geographic region.

In practice, each vehicle 24 of the fleet of delivery vehicles is outfitted with a sampling module 22. The vehicle 24 will then depart from a centralized location and make normal deliveries along a public road system. The sampling modules 22 then take biological samples of various instances. These instances can be random or adaptive modes, based upon time, can be based upon a predetermined amount of travel of the vehicle, or can be at programmable predetermined locations.

As mentioned previously, the chemical, radiological and/or biological sample is taken in addition to temporal information related to the location taken with each sample. The module includes a location-determining device such as a GPS system. It is envisioned that the location of each particular sample can also be determined based on triangulation of known local RF transmissions such as cell phone towers or computer network locations.

Additional information can be related to the time and date of a sample. Atmospheric information such as temperature humidity and/or wind speed can also be determined. It is envisioned that the sample can be taken while the vehicle is moving between two locations or can be taken when the vehicle is stopped. In this regard, the samples can be taken between a first time T₁ and a second time T₂ when the vehicle is moving, stopped, or combinations of the vehicle being moved or stopped. It is also envisioned that a single sample can be taken as the vehicle moves a predetermined distance from a first location L₁ to a second location L₂.

As described below, the sampling system uses an indexable filter media 28. The indexable filter media 28 is configured to allow the acquisition of multiple separate discrete chemical, radiological and/or biological samples over a specific time. Upon return of the vehicle to a centralized location, the filter media can be removed from the system and can be shipped via postal service to a central facility where the samples from each discrete location can be interrogated using PCR or real-time PCR processes to determine if the biological marker can be found within the sample. In the event chemical constituents are being monitored, testing apparatus such as mass spectrometry can be used.

As is known, biological markers for specific biological species are readily identifiable within a sample by use of individualized primers associated with the biological marker. These primers can be, by way of non-limiting example, designed to detect the presence of pollen from a genetically modified organism such RoundUp Ready™ grain crops. Additionally, primers can be used to determine if genetic material from the spores of various Myco infecting organisms are present at a specific location. This information is critically important as it can be used to determine which fungicides or herbicides may be effectively used to increase crop outputs and yield.

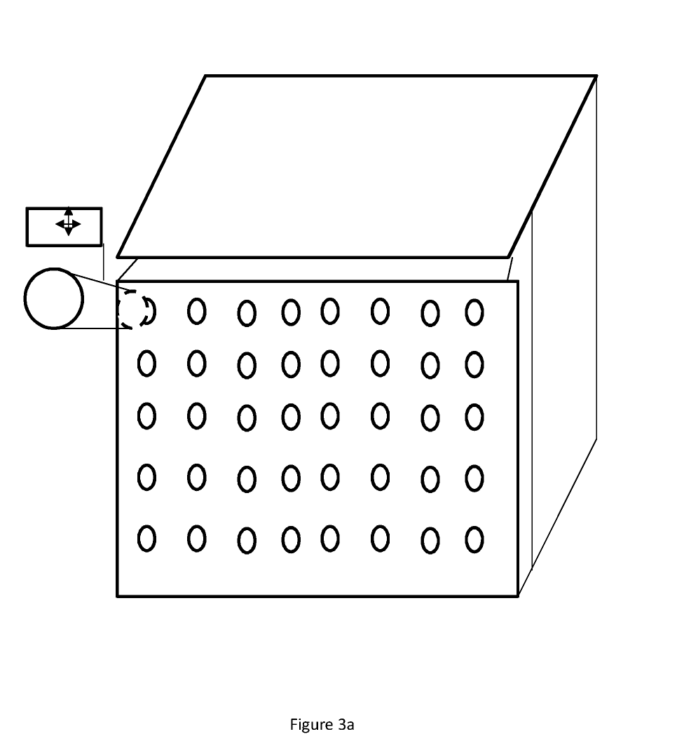

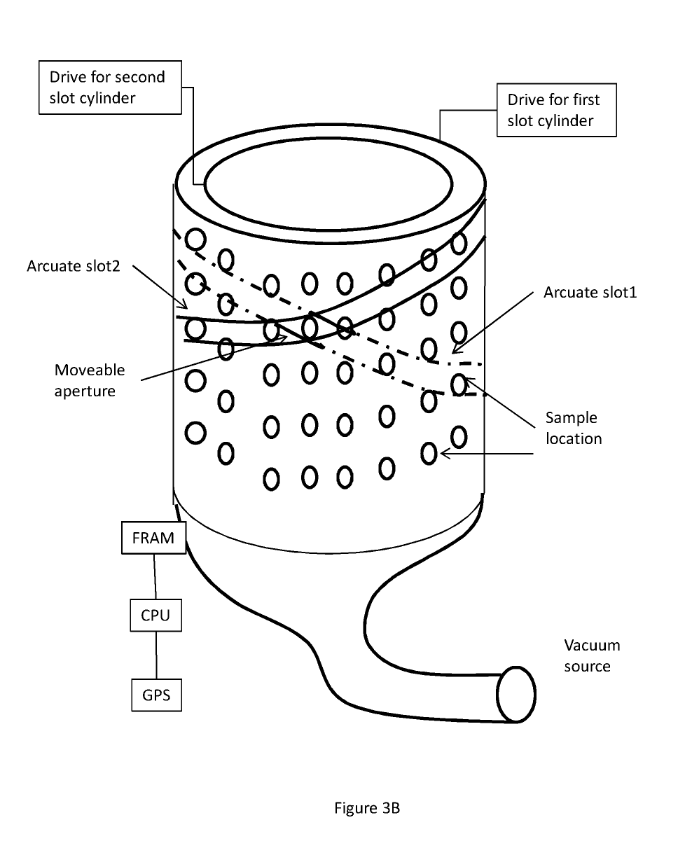

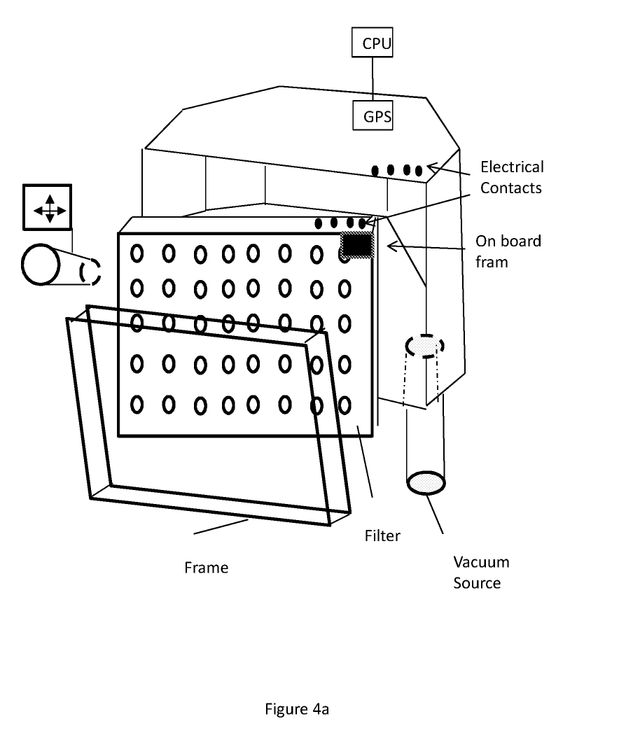

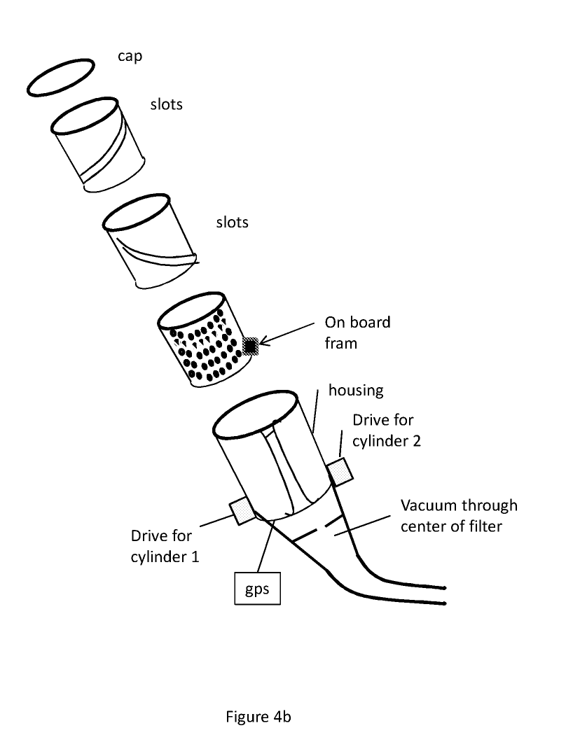

FIGS. 3a and 3b represent schematic views of the biological sampling module 22. Shown is a filter media 30 disposed adjacent to an indexable aperture 32. Indexable aperture 32 is configured to move in relation to the filter media 30 to allow the capture of biological or particulate matter within the filter at discrete sampling times, locations, or combinations thereof. In this regard, it is envisioned the filter media 30 can be moveable with respect to the aperture 32 or the aperture 32 can be moveable with respect to the filter media 30.

The filter media 30 is configured to capture biological materials which are found in a stream of air or liquid being passed through the indexable aperture 32. It is envisioned that the filter be configured to collect material larger than about 5 μm. A pre-filter which collects material larger than about 100 μm can be placed in the fluid stream before the stream reaches the media 30 to reduce the clogging of the filter media 30. Because discrete samples are being retrieved, the risk of fouling of the filter is minimized.

The filter media 30 can be planar or cylindrical. In this configuration, the indexable aperture 32 is configured to move in relation to the filter media 30 to provide an array of discrete sample locations 36 on the filter media 30. Locational information can be collected from the GPS positioning system and stored in an electronic memory. In situations when the sample is taken from a moving platform, an array of locational information can be taken and associated with a specific discrete sample 36. This information is stored in an associated computer storage data structure.

A vacuum source 40 is provided which draws air in a controlled and measurable fashion through the indexable aperture 32 and through a discrete location on the filter media 30. This vacuum source 40 can be, for example, an electrically powered vacuum, which is powered by the electrical system of the mobile platform. Optionally, the vacuum system 40 can be associated with the air intake of the vehicle.

In addition to the indexable system, associated with the filter media 30 can be a single sample filter location 44 with an individual separate non-indexable aperture 42. This single sample location can provide a summation of the entirety of a sample collection. In practice, air samples can simultaneously be taken through both the indexable aperture 32 and the non-indexable aperture 42. It is envisioned the second sample can be on a different filter media or a discrete media location on the indexable media. Should an investigator wish to determine if a single species of organism is present within the entire set of a sample, the collected sample 44 associated with the non-indexable aperture 42 can be first processed through a PCR or real time PCR process. A positive test result would indicate the detection of the biological marker along the entire vehicle's route, which has been recorded during the travel.

Should the desired biological or chemical marker be detected along the travel of the vehicle, each of the individual samples and filter locations associated with the indexable aperture can be processed through the PCR process to determine the exact location or locations where the biological markers were found among all of the discrete samples taken. Should real-time PCR be processed on the sample, location and signal strength or quantitative amount of the biological markers can be determined.

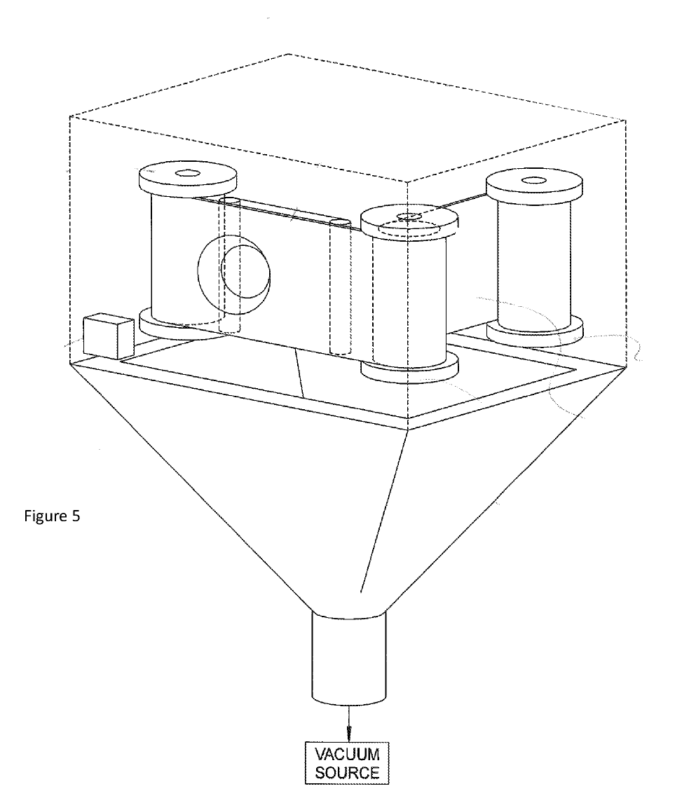

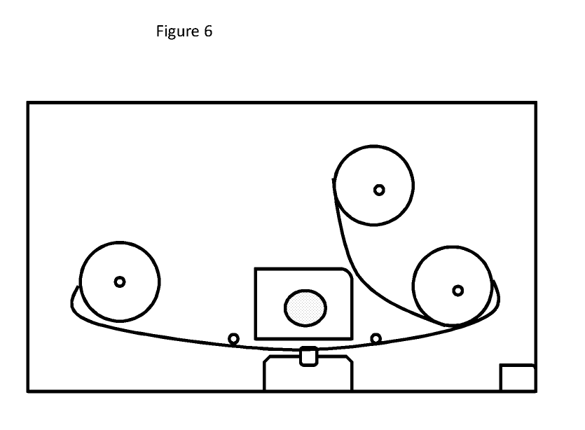

FIGS. 5 and 6 represent an alternate filter module 50 which utilize a filter media 30 that is moveable with respect to a fixed location aperture 52. The filter media 30 is suspended within a fluid or air path on a pair of rotatable spools 54. The spools 54 are rotated to position individual collection positions of the filter media 30 in front of the aperture 52.

A support member 56 can be positioned in contact with a back surface of the filter media 30 to support the filter in the presence of the air or fluid flow. A vacuum source as described above is associated with a housing 58 to draw airflow through the filter media 30 as described above. As described above, a non-moveable portion of the filter media 30 can be associated with a second non-indexable aperture 42 to allow a single test to be used for the summation characterization of the entire route (as described above). It is envisioned a second layer of protective film 64 can be used to prevent cross contamination of the filter media.

In this regard, each sampling module can have an associated CPU, power supply, I/O, and memory. The memory can be a stable FRAM memory. The locational information from the GPS system can be stored in a memory chip associated with the filter housing. This chip can be a FRAM construction. In this regard, the GPS and vacuum system can be semi-permanently fixed to the mobile platform, while the filter media and associated housing and memory can be a removable module. The entire removable module including the locational atmosphere and time information can be sent via overnight post to a central testing facility for analysis. The samples can then be analyzed as described above to determine if biological markers can be found with the non-moveable aperture. Should the biological markers be detected, the array of sample associated with the moveable filter can be individually interpreted to determine the exact geographic location or locations of the detection.

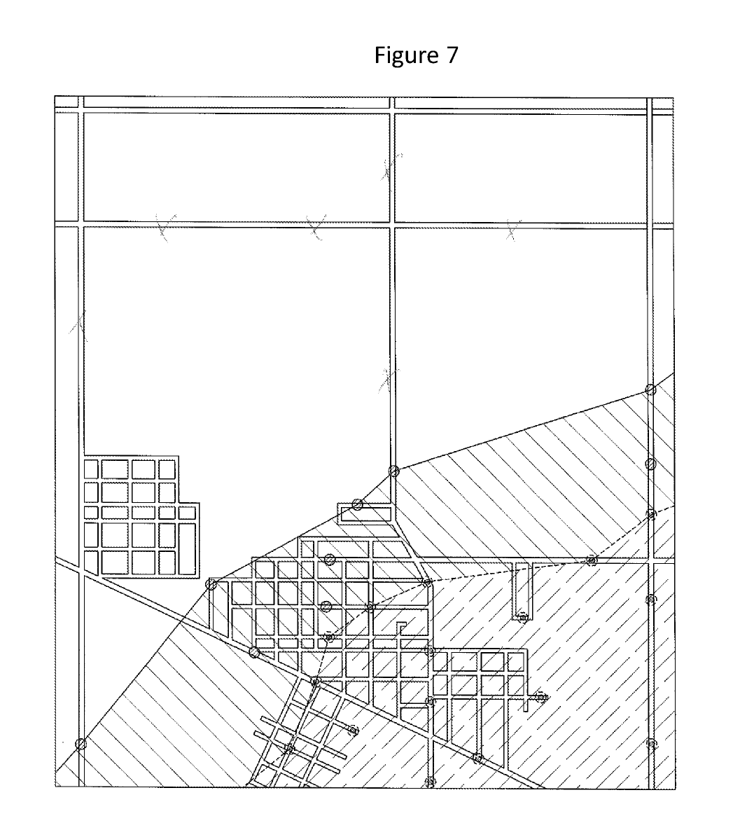

FIG. 7 represents a map showing the acquisition and detection of biological markers using the system described above. It is envisioned a fleet or a single mobile platform travels through the geographic region depicted on the map. Shown is a plurality of first points indicating the location of a sensed biological marker in the form of a detected DNA sequence. Also shown are locations where no biological Marker is detected. The dashed line is an approximate boundary for the transition of the boundary. It is envisioned the movement of this line over time can be used to measure and model the movement of a specific biologic material through the environment over time.

Because real-time PCR gives information related to the amount DNA of detected, the amount of biological material (in the form of an estimated number of pathogens, weight, or load) detected. Contour maps of the intensity of the biological markers can be seen on the map. Time and location data is collected, this date can be inputted into computer models or compared with results generated in computer models with regard to the spread of biological markers to help in the correlation of the models. These models can include wind, precipitation and temperature factors.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various implementations of the systems and techniques described here can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Implementations of the subject matter and the functional operations described in this specification can be implemented in digital electronic circuitry, or in computer software, firmware, or hardware, including the structures disclosed in this specification and their structural equivalents, or in combinations of one or more of them. Moreover, subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The terms “data processing apparatus”, “computing device” and “computing processor” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal, which is generated to encode information for transmission to suitable receiver apparatus.

A computer program (also known as an application, program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

One or more aspects of the disclosure can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for the distributed monitoring of biological markers within a geographic region comprising:
a plurality of mobile platforms distributed at locations within the geographic region;
a monitor associated with each of the plurality of mobile platforms, the monitor having an indexable air filter system configured to capture a plurality of individualized samples containing the biological markers at discrete locations within the filter medium;
a mobile platform geographic location sensor associated with each mobile platform; and
a controller for correlating the location of the plurality of individualized samples within the filter media with the location of an associated mobile platform geographic location.

2. The system according to claim 1, wherein the plurality of mobile platforms comprises at lease one wheeled ground vehicle.

3. The system according to claim 1, wherein the indexable filter system comprises a filter medium which is movable with respect to an air intake aperture.

4. The system according to claim 1, wherein the indexable filter system comprises a vacuum.

5. The system according to claim 1, wherein the filter medium is sized to collect fungal spores.

6. The system according to claim 1, wherein the filter medium is sized to collect pollen.

7. The system according to claim 1, wherein the geographic location sensor comprises a GPS.

8. The system according to claim 1, wherein the biological markers comprise DNA.

9. A system for the distributed monitoring of biological markers within a geographic region comprising:
a plurality of ground based mobile platforms movably distributed on a road system within the geographic region;
a vehicle monitoring system configured to record the location of the plurality of ground based mobile platforms between a first and a second time;
an air sampling system configured to capture a plurality of discrete samples containing biological markers, the air sampling system having a filter medium which is indexable with respect to an input aperture, each of the discrete samples being taken at locations on the filter medium; and
a controller configured to correlate specific locations of the vehicle in the geographic regions to at least one of the discrete samples.

10. The monitoring system according to claim 9, further comprising a display which displays the location of the mobile platforms within the geographic region.

11. The system according to claim 9, further comprising an actuator configured to move one of the aperture and the filter media with respect to each other.

12. The system according to claim 9, wherein the vehicle monitoring system comprises a GPS.

13. The system according to claim 9, further comprising an atmospheric state measurement system.

14. The system according to claim 13, wherein the atmospheric state measuring system measures at least one of temperature and humidity.

15. The system according to claim 9, wherein the plurality of mobile platforms is a fleet of wheeled vehicles.

16. The system according to claim 9, wherein the controller is configured to correlate specific locations of the vehicle to the discrete samples is located on a mobile platform.

17. The system according to claim 9, wherein the filter medium is configured to capture at least one of pollen and fungal spores.

18. The system according to claim 9, wherein the controller records the vehicle location and time when a sample is being taken by the air sampling system.

19. A method of detecting biological markers within a geographic region comprising:
providing a plurality of mobile platforms within the geographic region, each mobile platform having an air sampling system;
moving the plurality of mobile platforms within the geographic regions for a predetermined amount of time;
taking a first air sample at a first location in the geographic region to collect a first sample;
recording the location of the first location;
indexing a filter media in each of the plurality of air filtering systems;
taking a second air sample at a second location in the geographic location to collect a second sample;
recording the second location;
correlating the first sample to the first location; and
correlating the second sample with the second location.

20. The method according to claim 20, further comprising indexing the filter medium prior to taking a second air sample.
